(19) United States
(12) Patent Application Publication
(10) Pub. No.: US 2018/0235809 A1
(43) Pub. Date: Aug. 23, 2018

(54) TRANSITION ZONE SYSTEMS AND METHODS

(71) Applicant: AMO DEVELOPMENT LLC, Santa Ana, CA (US)

(72) Inventor: Guang-ming DAI, Fremont, CA (US)

(73) Assignee: AMO DEVELOPMENT LLC, Santa Ana, CA (US)

(21) Appl. No.: 15/900,634

(22) Filed: Feb. 20, 2018

Related U.S. Application Data
(60) Provisional application No. 62/462,192, filed on Feb. 22, 2017.

(51) Int. Cl. A61F 9/008; G16H 20/40

(52) U.S. Cl. CPC A61F 9/00804; G16H 20/40; A61F 2009/00842; A61F 2009/00882; A61F 2009/00872

(57) ABSTRACT

A method of re-profiling a cornea of an eye is provided which includes causing ablation energy to be applied across the cornea of the eye and controlling distribution of the ablation energy across the cornea of the eye. The distribution of the ablation energy is controlled by causing the ablation energy to provide an ablation zone, having an optical zone disposed in a central portion of the anterior surface and a transition zone disposed peripherally to the optical zone on an anterior surface of the cornea the ablation zone, and determining a shape of the transition zone by selecting between a cubic spline function and a complementary error function.

### CROSS-REFERENCES TO RELATED APPLICATIONS

This application claims priority to U.S. Provisional Patent Application No. 62/462,192, filed on Feb. 22, 2017, which is incorporated herein by reference.

### SUMMARY

A method of re-profiling a cornea of an eye is provided. The method includes causing ablation energy to be applied across the cornea of the eye and controlling distribution of the applied ablation energy across the cornea of the eye. The distribution of the applied ablation energy is controlled by causing the ablation energy to provide an ablation zone on an anterior surface of the cornea. The ablation zone includes an optical zone, disposed in a central portion of the anterior surface, and a transition zone, disposed peripherally to the optical zone, on the anterior surface. The distribution of the applied ablation energy is further controlled by determining a shape of the transition zone by selecting between a cubic spline function and a complementary error function.

A processing device for re-profiling a cornea of an eye is provided. The system includes memory, configured to store programmed instructions and data, and a processor in communication with the memory. The processor is configured to cause ablation energy to be applied across the cornea of the eye and to control distribution of the applied ablation energy across the cornea of the eye. The distribution of the applied ablation energy is controlled by causing the ablation energy to provide an ablation zone. The ablation zone includes an optical zone, disposed in a central portion of the anterior surface, and a transition zone, disposed peripherally to the optical zone, on an anterior surface of the cornea the ablation zone. The distribution of the applied ablation energy is further controlled by determining a shape of the transition zone by selecting between a cubic spline function and a complementary error function.

A system for re-profiling a cornea of an eye is provided. The system includes a laser energy source, configured to apply laser energy to ablate a cornea of the eye, and a processing device in communication with the laser energy source. The processing device includes memory, configured to store programmed instructions and data, and a processor in communication with the memory. The processor is configured to control the laser energy source to cause the laser energy to be applied across the cornea of the eye and control distribution of the applied laser energy across the cornea of the eye. The distribution of the laser energy is controlled by causing the laser energy to provide an ablation zone on an anterior surface of the cornea. The ablation zone includes an optical zone, disposed in a central portion of the anterior surface, and a transition zone disposed peripherally to the optical zone. The distribution of the applied laser energy is further controlled by determining a shape of the transition zone by selecting between a cubic spline function and a complementary error function.

### BRIEF DESCRIPTION OF THE DRAWINGS

A more detailed understanding can be had from the following description, given by way of example in conjunction with the accompanying drawings wherein:

FIG. 1 illustrates an example laser ablation system within which embodiments described herein may be implemented;

FIG. 2 illustrates an example of a computing environment within which embodiments described herein may be implemented;

FIG. 3A illustrates a wavefront measurement system within which embodiments described herein may be implemented;

FIG. 3B illustrates another wavefront measurement system within which embodiments described herein may be implemented;

FIG. 4A is a schematic illustration of an example laser ablation treatment according to embodiments described herein;

FIG. 4B is a schematic illustration of a front view of the example optical, transition, and ablation zones shown in FIG. 4A;

FIG. 5A is a graphical illustration of a cross section of a −4 D (diopter) ablation profile using a theoretical calculation from spline fitting and a cross section of a −4 D ablation profile for a 6 mm optical zone and a 9 mm ablation zone without transition zone fitting;

FIG. 5B is a blown up view of a portion of FIG. 5 illustrating the comparison between the cross sections shown in FIG. 5A;

FIG. 5C is a graphical illustration of cross sections of 5 exemplary −4 D treatment targets;

FIG. 5D is a blown up view of a portion of FIG. 5C illustrating the comparison between the different cross sections;

FIG. 5E is a graphical illustration comparing a cross section of a −4 D ablation profile using spline fitting and a cross section of a −4 D ablation profile for providing a transition zone according to an embodiment described herein;

FIG. 6 is a graphical illustration of an exemplary complementary error function and its first derivative for use with embodiments described herein;

FIG. 7 is a graphical illustration of normalized and non-normalized derivatives of a complementary error function for use with embodiments described herein;

FIG. 8 is a graphical illustration of cross-sections of exemplary ablation profiles for a spherical myopia refractive treatment;

FIG. 9 is a graphical illustration of cross-sections of exemplary ablation profiles for a myopic astigmatism refractive treatment;

FIG. 10 is a graphical illustration of cross-sections of exemplary ablation profiles for a mixed astigmatism refractive treatment;

FIG. 11 is a graphical illustration of an exemplary ablation profile for a myopic treatment with astigmatism having a transition zone on the major axis;

FIG. 12 is a graphical illustration of an exemplary ablation profile of a cylinder having a transition zone on the major axis;

FIG. 13 is a graphical illustration of an exemplary ablation profile for a mixed astigmatism treatment shape having a transition zone on the major axis;

FIG. 14 is a block diagram illustrating an exemplary method of re-profiling a cornea of an eye according to an embodiment described herein;

FIG. 15 is a block diagram illustrating an exemplary method of determining a transition zone according to an embodiment described herein; and

FIG. 16 is an illustration of different exemplary ablation shapes provided by different laser pulses for use with embodiments described herein.

### DETAILED DESCRIPTION OF THE PREFERRED EMBODIMENTS

Conventional laser-based systems and methods exist for enabling ophthalmic surgery on the cornea to treat vision defects, including myopia (nearsightedness), hyperopia (farsightedness), and symmetrical cylindrical astigmatisms. For example, in patients with myopia, the focusing power of the cornea (and/or the lens) can be too high, such that light entering the eye is focused at a location anterior to the retina. Treatments for myopia typically strive to place the focus point of the incoming light at the surface of the retina. Hence, if the curvature of the patient cornea is too high, the treatment can involve reducing the curvature of the cornea.

These conventional vision treatment systems and methods typically include ablative photodecomposition, which selectively exposes the cornea to laser radiation to remove a microscopic layer of stromal tissue from the cornea. The ablation leads to a resculpting of the cornea, without causing significant thermal damage to adjacent and underlying tissues of the eye. Corneal shaping is intended to change the optical properties of an eye, and thus treat optical defects, such as refractive errors. Such shaping is often performed in stromal tissue of the cornea, while a flap of overlying tissue is temporarily displaced in a procedure known as Laser In Situ Keratomileusis (LASIK).

The distribution of ablation energy across the cornea can be controlled using different system components and methods, including ablatable masks, fixed and moveable apertures, controlled scanning systems and eye movement tracking mechanisms. The laser beam often comprises a series of discrete pulses of laser light energy, with the resulting corneal shape and amount of tissue removed being determined by factors, such as for example, shape, size, location, or the number of laser energy pulses impinging on the cornea. A variety of software and hardware combinations may be used to generate the pattern of laser pulses that reshape the cornea. Various forms of lasers and laser energies can be used to effect the treatment, such as for example, infrared lasers, ultraviolet lasers, femtosecond lasers and wavelength multiplied solid-state lasers.

Ablation profiles which have an abrupt periphery can be modified to provide a transition zone between the optical zone and the surrounding corneal surface to avoid resulting discontinuities in the ablated surface. Cubic spline functions work well for implementing transition zones in refractive surgical types such as hyperopia and hyperopic astigmatism. For conditions such as myopia, myopic astigmatism, and mixed astigmatism, however, in which transition zones can include negative values, cubic spline functions are not adequate.

Embodiments described herein include developing and implementing transition zones for use in laser eye surgery, and in particular, selectively ablating corneal tissue to improve the vision of patients having corneal irregularities or other vision defects, such as myopia, myopic astigmatism, and mixed astigmatism. Embodiments facilitate the avoidance of abrupt changes in ablation depth of the ablation profile, particularly toward the peripheral areas of the ablation. Embodiments described herein utilize a complementary error function for developing the transition zone curve for different refractive types, gaining the benefit of analytical expression of its derivatives and non-negativity.

Embodiments disclosed herein treat myopic conditions by laser sculpting corneal tissue to reduce the curvature of the cornea, such as by providing an ablation profile having an ablation depth that decreases with distance from the intended center of ablation, resulting in a substantially spherical ablated shape for the cornea, with decreased curvature, and with a lower or minimum depth of cut at or toward the outer edge of the optically correct portion of the ablation zone. Embodiments may be particularly useful for myopic meridians with a surface slope less than −2.

In addition to ablating human corneal tissue, the systems and methods described herein are well suited for ablating a wide variety of materials, such as plastic, polymethylacrylate (PMMA), porcine and bovine corneal tissue, and the like.

Embodiments disclosed herein are particularly useful for enhancing the accuracy and efficacy of laser eye surgical procedures, such as photorefractive keratectomy (PRK), phototherapeutic keratectomy (PTK), laser in situ keratomileusis (LASIK), laser assisted epithelium keratomileusis (LASEK), and the like. Embodiments described herein provide enhanced refractive procedures by improving the methodology for deriving or generating a corneal ablation profile.

Although the system and methods disclosed herein are primarily described in the context of a laser eye surgery system, it should be understood that the techniques described herein may be adapted for use in alternative eye treatment procedures and systems such as radial keratotomy (e.g., by attenuating an incision depth at the periphery of a radial keratotomy incision), intraocular lenses, collagenous corneal tissue thermal remodeling, removable corneal lens structures, and the like.

Although embodiments disclosed herein are described primarily in the context of a laser eye surgery system, it should be understood embodiments may be adapted for use in alternative eye treatment procedures and systems such as spectacle lenses, intraocular lenses, contact lenses, corneal ring implants, collagenous corneal tissue thermal remodeling, and the like.

Exemplary systems and methods disclosed herein can be implemented via a variety of ophthalmic devices or solutions. For example, treatment techniques may be used for any of a variety of surgery modalities, including excimer laser surgery, femtosecond surgery, and the like. A variety of forms of lasers and laser energy can be used to effect a correction or treatment, including infrared lasers, ultraviolet lasers, femtosecond lasers, wavelength multiplied solid-state lasers, and the like. By way of non-limiting example, ophthalmic corrections can involve a cornea or lens reshaping procedure, such as, for example using a picosecond or femtosecond laser. Laser ablation procedures can remove a targeted amount stroma of a cornea to change a cornea's contour and adjust for aberrations.

In some embodiments, a treatment protocol can involve the delivery of a series of discrete pulses of laser light energy, with a total shape and amount of tissue removed being determined by a shape, size, location, and/or number of laser energy pulses impinging on or focused within a cornea.

In some embodiments, a surgical laser, such as a non-ultraviolet, ultra-short pulsed laser that emits radiation with pulse durations as short as nanoseconds and femtoseconds (e.g., a femtosecond laser, or a picosecond laser) can be used to treat the eye of a patient. Other pulse widths may be suitable as well. The laser systems can be configured to deliver near infrared light. Other wavelengths may be used as well.

The laser systems can be configured to deliver laser light focused at a focus depth (e.g. within corneal or other ophthalmologic tissue) which may be controlled by the system. Laser surgery with ultra-short pulse lasers such as femtosecond lasers can be used to treat the eye. These pulsed lasers can make very accurate incisions of the eye and can be used in many ways to treat the eye. Additional types of incisions that can be performed with the short pulse lasers include incisions for paracentesis, limbal relaxing incisions, and refractive incisions to shape the cornea, for example.

In some embodiments, vision treatments can include focusing femtosecond laser energy within the stroma so as to ablate a volume of intrastromal tissue. By scanning the focal spot within an appropriate volume of the stromal tissue, it is possible to vaporize the volume so as to achieve a desired refractive alteration. Accordingly laser surgical techniques that involve femtosecond laser photodisruption or photoalteration treatments may be used according to embodiments disclosed herein. In some aspects, a femtosecond laser can be used to perform the photodisruption, thus providing an easy, precise, and effective approach to refractive surgery.

According to some embodiments, a femtosecond laser (or other laser) of the optical system can be used to incise the cornea or to cut a flap. A femtosecond laser may be used to make arcuate or other incisions in the cornea. The incisions may be customized, intrastromal, stable, predictable, and the like. Likewise, corneal entry incisions may be made, which are custom, multi-plane, and self-sealing.

Pulsed laser beams include bursts or pulses of light. Pulsed lasers, such as non-ultraviolet, ultra-short pulsed lasers with pulse durations measured in the nanoseconds to femtoseconds range, can be used in ophthalmic surgical procedures as disclosed herein. For example, a pulsed laser beam can be focused onto a desired area of ophthalmologic material or tissue, such as the cornea, the capsular bag, or the lens of the eye, to photoalter the material in this area and, in some instances, the associated peripheral area. Examples of photoalteration of the material include, but are not necessarily limited to, chemical and physical alterations, chemical and physical breakdown, disintegration, ablation, photodisruption, vaporization. Exemplary treatment systems can include a focusing mechanism (e.g. lens) and/or a scanning mechanism so as to guide or direct a focus of femtosecond energy along a path within the patient's eye (e.g. at one or more corneal subsurface locations).

According to some embodiments, the systems and methods disclosed herein can be implemented in connection with software residing in a diagnostic device such as WaveScan® and iDesign™ devices.

The broad beam top hat laser profile of ablation systems such as the STAR S4® Excimer Laser System by Abbott Medical Optics Inc. is highly effective in ablating myopic shapes, due to the high efficiency of material removal in unit time. Similar efficiencies can be achieved for the ablation of hyperopic shapes. For example reducing the maximum spot size from 6.5 mm to about 4 mm, can effectively reducing the maximum efficiency to 42/6.52=38%. Furthermore, the solution accuracy tolerance, which may be defined as the root mean squares (RMS) error between a target shape and an ablated shape, can involve the use of more small pulses, bringing such an efficiency reduction in practice to the level of nearly 15% for hyperopia. For example, a typical −4 D (diopters) treatment may involve an ablation of 20 seconds, and a typical +4 D (diopters) treatment may involve an ablation of 120 seconds using a 20 Hz laser. The use of other ablation shapes optionally combined with basis data adjustment techniques can improve the treatment time for hyperopia and other vision conditions.

Embodiments disclosed herein can be readily adapted for use with existing laser systems and other optical treatment devices. Although system, software, and method embodiments described herein are described primarily in the context of a laser eye surgery system, it should be understood that embodiments described herein may be adapted for use in alternative eye treatment procedures, systems, or modalities, such as spectacle lenses, intraocular lenses, accommodating IOLs, contact lenses, corneal ring implants, collagenous corneal tissue thermal remodeling, corneal inlays, corneal onlays, other corneal implants or grafts, and the like. Relatedly, systems, software, and methods according to embodiments described herein are well suited for customizing any of these treatment modalities to a specific patient. Thus, for example, embodiments encompass custom intraocular lenses, custom contact lenses, custom corneal implants, and the like, which can be configured to treat or ameliorate any of a variety of vision conditions in a particular patient based on their unique ocular characteristics or anatomy. Additionally, the ablation target or target shape may be implemented via other non-ablative laser therapies, such as laser-incised custom lenticule shapes and subsequent extraction and laser-based corneal incision patterns.

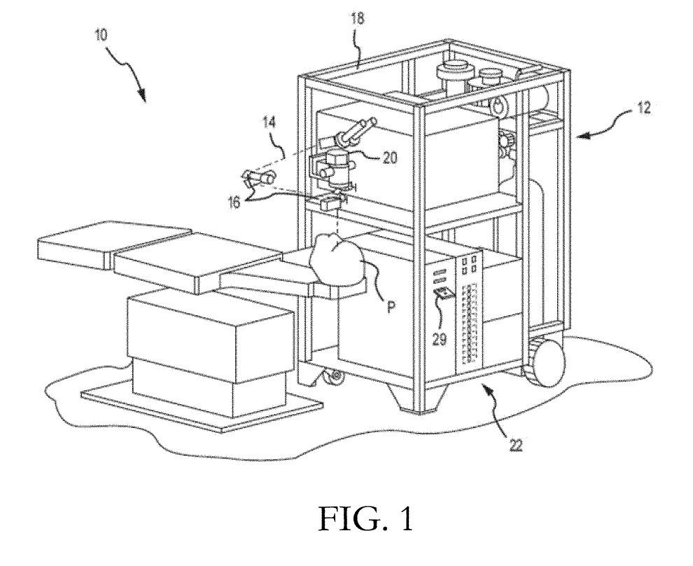

Turning now to the drawings, FIG. 1 illustrates a laser eye surgery system 10, including a laser 12 that produces a laser beam 14. Laser 12 is optically coupled to laser delivery optics 16, which directs laser beam 14 to an eye E of patient P. A delivery optics support structure (not shown here for clarity) extends from a frame 18 supporting laser 12. A microscope 20 is mounted on the delivery optics support structure, the microscope often being used to image a cornea of eye E.

Laser 12 generally comprises an excimer laser, ideally comprising an argon-fluorine laser producing pulses of laser light having a wavelength of approximately 193 nm. Laser 12 will preferably be designed to provide a feedback stabilized fluence at the patient's eye, delivered via delivery optics 16. Embodiments described herein may also be useful with alternative sources of ultraviolet or infrared radiation, particularly those adapted to controllably ablate the corneal tissue without causing significant damage to adjacent and/or underlying tissues of the eye. Such sources include, but are not limited to, solid state lasers and other devices which can generate energy in the ultraviolet wavelength between about 185 and 205 nm and/or those which utilize frequency-multiplying techniques. Hence, although an excimer laser is the illustrative source of an ablating beam, other lasers may be used.

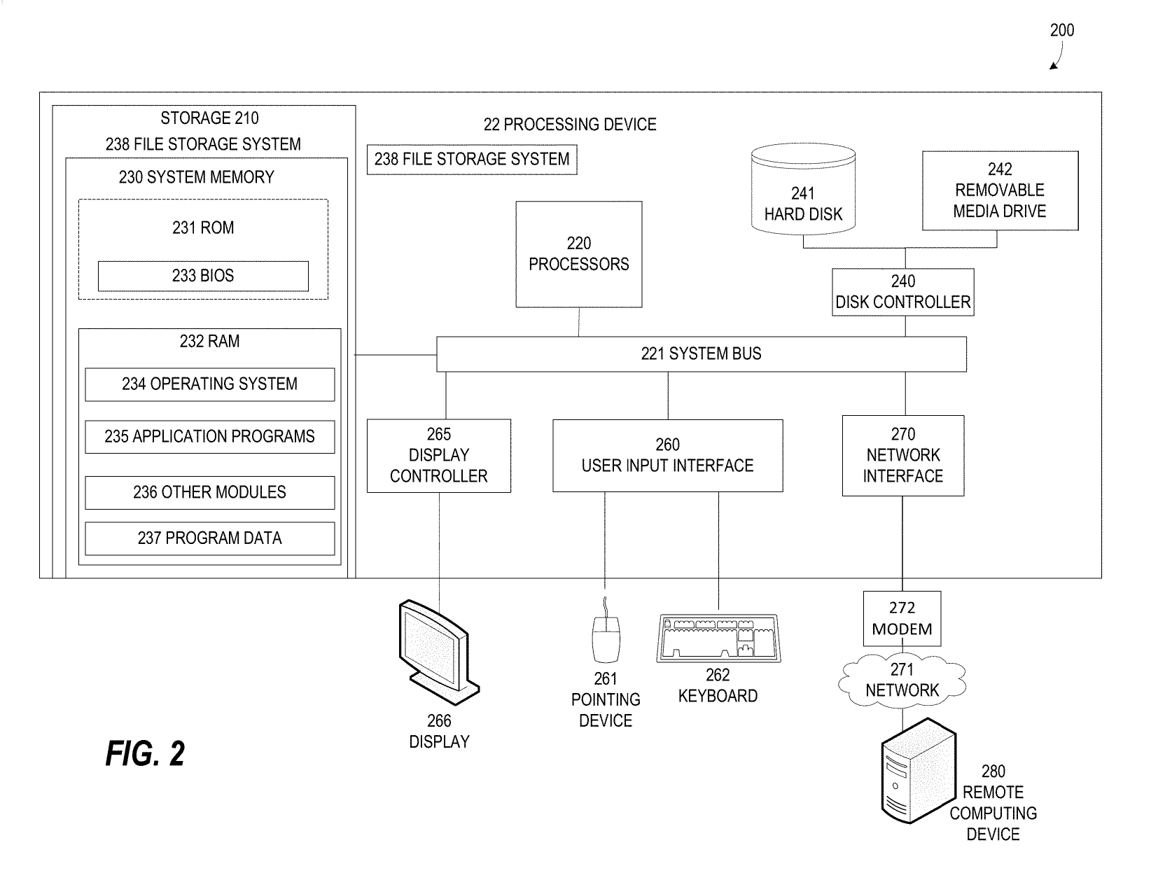

The exemplary laser system 10 includes processing device 22 and tangible storage media 29, described in more detail below with regard to FIG. 2. Laser 12 and delivery optics 16 will generally direct laser beam 14 to the eye of patient P under the direction of processing device 22. Processing device 22 will often selectively adjust laser beam 14 to expose portions of the cornea to the pulses of laser energy so as to effect a predetermined sculpting of the cornea and alter the refractive characteristics of the eye. In many embodiments, both laser beam 14 and the laser delivery optical system 16 are controlled by processing device 22 to effect the desired laser sculpting process, with the processing device 22 effecting (and optionally modifying) the pattern of laser pulses. The pattern of pulses may by summarized in machine readable data of tangible storage media 29 in the form of a treatment table, and the treatment table may be adjusted according to feedback input into processing device 22 from an automated image analysis system in response to feedback data provided from an ablation monitoring system feedback system.

Optionally, the feedback may be manually entered into a processor by a system operator. Such feedback might be provided by integrating the wavefront measurement system described below with the laser treatment system 10, and processing device 22 may continue and/or terminate a sculpting treatment in response to the feedback, and may optionally also modify the planned sculpting based at least in part on the feedback. Measurement systems are further described in U.S. Pat. No. 6,315,413, the full disclosure of which is incorporated herein by reference.

Laser beam 14 may be adjusted to produce the desired sculpting using a variety of alternative mechanisms. The laser beam 14 may be selectively limited using one or more variable apertures. An exemplary variable aperture system having a variable iris and a variable width slit is described in U.S. Pat. No. 5,713,892, the full disclosure of which is incorporated herein by reference. The laser beam may also be tailored by varying the size and offset of the laser spot from an axis of the eye, as described in U.S. Pat. Nos. 5,683,379, 6,203,539, and 6,331,177, the full disclosures of which are incorporated herein by reference.

Still further alternatives are possible, including: scanning of the laser beam over the surface of the eye and controlling the number of pulses and/or dwell time at each location, as described, for example, by U.S. Pat. No. 4,665,913, the full disclosure of which is incorporated herein by reference; using masks in the optical path of laser beam 14 which ablate to vary the profile of the beam incident on the cornea, as described in U.S. Pat. No. 5,807,379, the full disclosure of which is incorporated herein by reference; hybrid profile-scanning systems in which a variable size beam (typically controlled by a variable width slit and/or variable diameter iris diaphragm) is scanned across the cornea; or the like.

Additional components and subsystems may be included with laser system 10, as should be understood by those of skill in the art. For example, spatial and/or temporal integrators may be included to control the distribution of energy within the laser beam, as described in U.S. Pat. No. 5,646,791, the full disclosure of which is incorporated herein by reference. Ablation effluent evacuators/filters, aspirators, and other ancillary components of the laser surgery system are known in the art. Further details of suitable systems for performing a laser ablation procedure can be found in commonly assigned U.S. Pat. Nos. 4,665,913, 4,669,466, 4,732,148, 4,770,172, 4,773,414, 5,207,668, 5,108,388, 5,219,343, 5,646,791 and 5,163,934, the complete disclosures of which are incorporated herein by reference. Suitable systems also include commercially available refractive laser systems such as those manufactured and/or sold by Alcon, Bausch & Lomb, Nidek, WaveLight, LaserSight, Schwind, Zeiss-Meditec, and the like. Basis data can be further characterized for particular lasers or operating conditions, by taking into account localized environmental variables such as temperature, humidity, airflow, and aspiration.

FIG. 2 illustrates an example of a computing environment 200 within which embodiments described herein may be implemented. Computing environment 200 may be implemented as part of any component described herein. As shown in FIG. 2, computing environment 200 includes processing device 22 shown in FIG. 1, which is one example of a processing device in which embodiments may be implemented. As shown in FIG. 2, the processing device 22 may include a communication mechanism such as a bus 221 or other communication mechanism for communicating information within the processing device 22. The system 210 further includes one or more processors 220 (hereinafter “processors”) coupled with the bus 221 for processing the information. The processors 220 may include one or more CPUs, GPUs, or any other processor.

The processing device 22 also includes storage 210, including system memory 230 and file storage system 238, which is coupled to the bus 221 for storing information and instructions to be executed by processors 220. The system memory 230 may include computer readable storage media in the form of volatile and/or nonvolatile memory, such as read only memory (ROM) 231 and/or random access memory (RAM) 232. The system memory RAM 232 may include other dynamic storage device(s) (e.g., dynamic RAM, static RAM, and synchronous DRAM). The system memory ROM 231 may include other static storage device(s) (e.g., programmable ROM, erasable PROM, and electrically erasable PROM). In addition, the system memory 230 may be used for storing temporary variables or other intermediate information during the execution of instructions by the processors 220. A basic input/output system 233 (BIOS) containing the basic routines that help to transfer information between elements within processing device 22, such as during start-up, may be stored in ROM 231. RAM 232 may contain data and/or program modules that are immediately accessible to and/or presently being operated on by the processors 220. System memory 230 may additionally include, for example, operating system 234, application programs 235, other program modules 236 and program data 237.

The processing device 22 also includes a disk controller 240 coupled to the bus 221 to control one or more storage devices for storing information and instructions, such as a magnetic hard disk 241 and a removable media drive 242 (e.g., floppy disk drive, compact disc drive, tape drive, and/or solid state drive). The storage devices may be added to the processing device 22 using an appropriate device interface (e.g., a small computer system interface (SCSI), integrated device electronics (IDE), Universal Serial Bus (USB), or FireWire).

The processing device 22 may also include a display controller 265 coupled to the bus 221 to control a display or monitor 266, such as a cathode ray tube (CRT) or liquid crystal display (LCD), for displaying information to a computer user. The processing device 22 includes a user input interface 260 and one or more input devices, such as a keyboard 262 and a pointing device 261, for interacting with a computer user and providing information to the processors 220. The pointing device 261, for example, may be a mouse, a trackball, or a pointing stick for communicating direction information and command selections to the processor 220 and for controlling cursor movement on the display 266. The display 266 may provide a touch screen interface which allows input to supplement or replace the communication of direction information and command selections by the pointing device 261.

The processing device 22 may perform a portion or all of the processing steps of embodiments described herein in response to the processors 220 executing one or more sequences of one or more instructions contained in a memory, such as the system memory 230. Such instructions may be read into the system memory 230 from another computer readable medium, such as a hard disk 241 or a removable media drive 242. The hard disk 241 may contain one or more data stores and data files used by embodiments described herein. Data store contents and data files may be encrypted to improve security. The processors 220 may also be employed in a multi-processing arrangement to execute the one or more sequences of instructions contained in system memory 230. In alternative embodiments, hard-wired circuitry may be used in place of or in combination with software instructions. Thus, embodiments are not limited to any specific combination of hardware circuitry and software.

As stated above, the processing device 22 may include at least one computer readable medium or memory for holding instructions programmed according to embodiments described herein and for containing data structures, tables, records, or other data described herein. The term “computer readable medium” as used herein refers to any non-transitory, tangible medium that participates in providing instructions to the processor 220 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 241 or removable media drive 242, such as storage media 29 shown in FIG. 1. Removable media drive 242 may optionally embody wavefront sensor data, wavefront gradients, a wavefront elevation map, a treatment map, a corneal elevation map, and/or an ablation table. Non-limiting examples of volatile media include dynamic memory, such as system memory 230. Non-limiting examples of transmission media include coaxial cables, copper wire, and fiber optics, including the wires that make up the bus 221. Transmission media may also take the form of acoustic or light waves, such as those generated during radio wave and infrared data communications.

The computing environment 200 may further include the processing device 22 operating in a networked environment using logical connections to one or more remote computers, such as remote computer 280. Remote computer 280 may be a personal computer (laptop or desktop), a mobile device, a server, a router, a network PC, a peer device or other common network node, and typically includes many or all of the elements described above relative to computer 210. When used in a networking environment, computer 210 may include modem 272 for establishing communications over a network 271, such as the Internet. Modem 272 may be connected to system bus 221 via network interface 270, or via another appropriate mechanism.

Network 271 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between processing device 22 and other computers (e.g., remote computing system 280). The network 271 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 271.

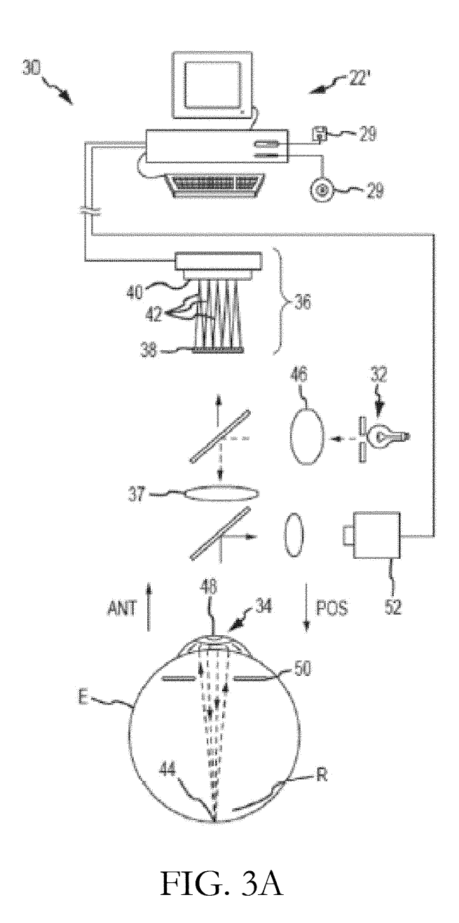

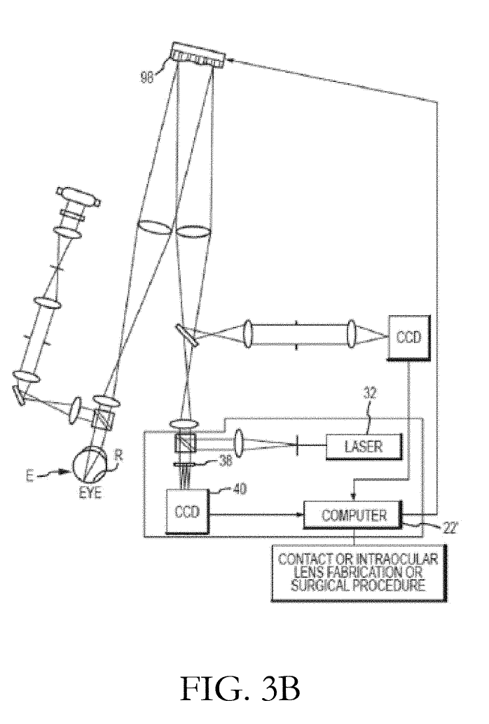

FIG. 3A is a schematic illustration of a wavefront measurement system 30 which may be used according to embodiments described herein. Wavefront measurement system 30 is configured to sense local slopes of a gradient map exiting the patient's eye. Devices based on the Hartmann-Shack principle generally include a lenslet array to sample the gradient map uniformly over an aperture, which is typically the exit pupil of the eye. Thereafter, the local slopes of the gradient map are analyzed so as to reconstruct the wavefront surface or map.

Wavefront measurement system 30 includes an image source 32, such as a laser, which projects a source image through optical tissues 34 of eye E so as to form an image 44 upon a surface of retina R. The image from retina R is transmitted by the optical system of the eye (e.g., optical tissues 34) and imaged onto a wavefront sensor 36 by system optics 37. The wavefront sensor 36 communicates signals to processing device 22′, for measurement of the optical errors in the optical tissues 34 and/or determination of an optical tissue ablation treatment program. Wavefront measurement system 30 also includes another processing device 22′, which may include the same or similar components as the processing device 22 illustrated in FIGS. 1 and 2. In some embodiments, processing device 22′ may be in communication with processing device 22, which directs the laser surgery system 10, or some or all of the components of processing device 22. Processing device 22 and processing device 22′ may be integrated or separate from each other. Data from wavefront sensor 36 may be transmitted to processing device 22 via tangible media 29, via an I/O port, via network interface 270, such as an intranet or the Internet, or the like.

Wavefront sensor 36 generally comprises a lenslet array 38 and an image sensor 40. As the image from retina R is transmitted through optical tissues 34 and imaged onto a surface of image sensor 40 and an image of the eye pupil P is similarly imaged onto a surface of lenslet array 38, the lenslet array separates the transmitted image into an array of beamlets 42, and (in combination with other optical components of the system) images the separated beamlets on the surface of sensor 40. Sensor 40 typically comprises a charged couple device or “CCD,” and senses the characteristics of these individual beamlets, which can be used to determine the characteristics of an associated region of optical tissues 34. In particular, where image 44 comprises a point or small spot of light, a location of the transmitted spot as imaged by a beamlet can directly indicate a local gradient of the associated region of optical tissue.

Eye E generally defines an anterior orientation ANT and a posterior orientation POS. Image source 32 generally projects an image in a posterior orientation through optical tissues 34 onto retina R as indicated in FIG. 3A. Optical tissues 34 again transmit image 44 from the retina anteriorly toward wavefront sensor 36. Image 44 actually formed on retina R may be distorted by any imperfections in the eye's optical system when the image source is originally transmitted by optical tissues 34. Optionally, image source projection optics 46 may be configured or adapted to decrease any distortion of image 44.

In some embodiments, image source optics 46 may decrease lower order optical errors by compensating for spherical and/or cylindrical errors of optical tissues 34. Higher order optical errors of the optical tissues may also be compensated through the use of an adaptive optic element, such as a deformable mirror (described below). Use of an image source 32 selected to define a point or small spot at image 44 upon retina R may facilitate the analysis of the data provided by wavefront sensor 36. Distortion of image 44 may be limited by transmitting a source image through a central region 48 of optical tissues 34 which is smaller than a pupil 50, as the central portion of the pupil may be less prone to optical errors than the peripheral portion. Regardless of the particular image source structure, it will be generally be beneficial to have a well-defined and accurately formed image 44 on retina R.

In one embodiment, the wavefront data may be stored in a computer readable medium 29 or a memory of the wavefront sensor system 30 in two separate arrays containing the x and y wavefront gradient values obtained from image spot analysis of the Hartmann-Shack sensor images, plus the x and y pupil center offsets from the nominal center of the Hartmann-Shack lenslet array, as measured by the pupil camera 51 (FIG. 3A) image. Such information contains all the available information on the wavefront error of the eye and is sufficient to reconstruct the wavefront or any portion of it. In such embodiments, there is no need to reprocess the Hartmann-Shack image more than once, and the data space required to store the gradient array is not large. For example, to accommodate an image of a pupil with an 8 mm diameter, an array of a 20×20 size (i.e., 400 elements) is often sufficient. As can be appreciated, in other embodiments, the wavefront data may be stored in a memory of the wavefront sensor system in a single array or multiple arrays.

While the methods will generally be described with reference to sensing of an image 44, it should be understood that a series of wavefront sensor data readings may be taken. For example, a time series of wavefront data readings may help to provide a more accurate overall determination of the ocular tissue aberrations. As the ocular tissues can vary in shape over a brief period of time, a plurality of temporally separated wavefront sensor measurements can avoid relying on a single snapshot of the optical characteristics as the basis for a refractive correcting procedure. Still further alternatives are also available, including taking wavefront sensor data of the eye with the eye in differing configurations, positions, and/or orientations. For example, a patient will often help maintain alignment of the eye with wavefront measurement system 30 by focusing on a fixation target, as described in U.S. Pat. No. 6,004,313, the full disclosure of which is incorporated herein by reference. By varying a position of the fixation target as described in that reference, optical characteristics of the eye may be determined while the eye accommodates or adapts to image a field of view at a varying distance and/or angles.

The location of the optical axis of the eye may be verified by reference to the data provided from a pupil camera 52. In the exemplary embodiment, a pupil camera 52 images pupil 50 so as to determine a position of the pupil for registration of the wavefront sensor data relative to the optical tissues.

An alternative embodiment of a wavefront measurement system is illustrated in FIG. 3B. The major components of the system of FIG. 3B are similar to those of FIG. 3A. Additionally, FIG. 3B includes an adaptive optical element 53 in the form of a deformable mirror. The source image is reflected from deformable mirror 98 during transmission to retina R, and the deformable mirror is also along the optical path used to form the transmitted image between retina R and imaging sensor 40. Deformable mirror 98 can be controllably deformed by computer system 22′ to limit distortion of the image formed on the retina or of subsequent images formed of the images formed on the retina, and may enhance the accuracy of the resultant wavefront data. The structure and use of the system of FIG. 3B are more fully described in U.S. Pat. No. 6,095,651, the full disclosure of which is incorporated herein by reference.

The components of an embodiment of a wavefront measurement system for measuring the eye and ablations may comprise elements of a WaveScan® System. One embodiment includes a WaveScan® System with a deformable mirror as described above. An alternate embodiment of a wavefront measuring system is described in U.S. Pat. No. 6,271,915, the full disclosure of which is incorporated herein by reference. It is appreciated that any wavefront aberrometer could be employed for use with embodiments disclosed herein.

Processor 52 is configured to control an ablation energy source to provide an exemplary laser ablation treatment as described herein. For example, processor 52 is configured to control an ablation energy source, such as laser 12, to apply ablation energy across a cornea of an eye. Although other ablation energy sources may be used, for simplification purposes, the embodiments are described with reference to laser 12.

The processor 52 is configured to distribute the ablation energy across the cornea by controlling laser 12 to apply a laser energy treatment. The laser 12 is controlled to ablate the cornea such that an ablation zone is created on an anterior surface of the cornea. The ablation zone includes an optically correct central optical zone disposed in a central portion of the anterior surface of the cornea and a transition zone disposed peripherally to the central optical zone. The transition zone is defined by a complementary error function.

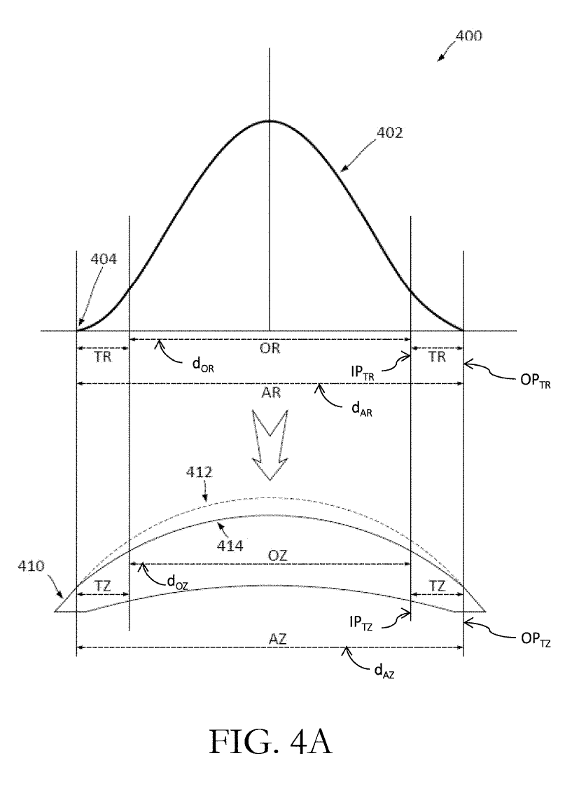

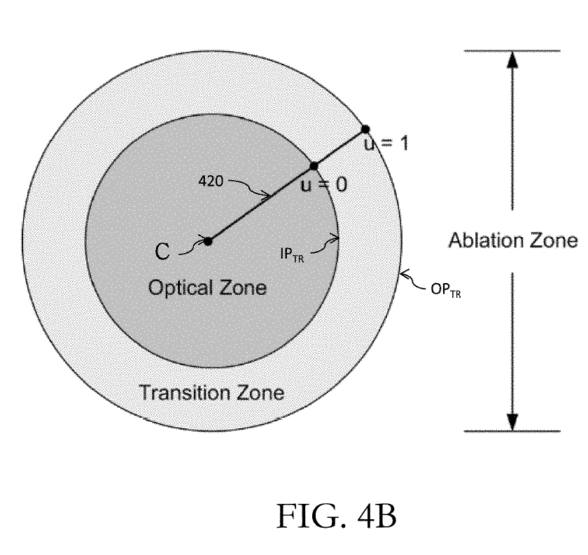

FIG. 4A is a schematic illustration of an exemplary laser ablation treatment 400, also referred to as a target treatment, in which the cornea is modified to a lower powered ablated contour. An ablation zone AZ is used herein to define the region of the cornea to be ablated. The location of the ablation zone AZ with respect to the pupil depends on the desired optical correction. In the embodiment shown in FIGS. 4A and 4B, the center C of the ablation zone AZ is located at the center of the pupil. Embodiments may, however, include an ablation zone AZ having a center which is not at the pupil's center. The optical zone OZ of the cornea typically corresponds to the area defined by a maximum pupil size of the eye, such as when the pupil is fully and completely dilated.

The top portion of FIG. 4A shows an exemplary laser ablation profile 402 illustrating Gaussian beam intensity defined by an ablation region AR. The bottom portion of FIG. 4A illustrates an ablation zone AZ, on a cornea 410 of a patient eye, which corresponds to ablation region AR of laser ablation profile 402. As shown in FIG. 4A, the ablation region AR includes a central optical region OR and a transition region TR, which is disposed peripheral to the central optical region OR.

Administration of the laser ablation profile 402 to the patient eye operates to ablate and reshape the cornea 410. Specifically, the central optical region OR of the ablation profile 402 produces an optical zone OZ on the patient cornea 410 and the transition region TR of the ablation profile 402 produces a transition zone TZ on the patient cornea, peripheral to the central optical zone OZ. In this way, the transition zone TZ provides a transition or connection between the optical zone OZ and the untreated or unablated portion of the cornea 410.

In some embodiments, the optical region OR or optical zone OZ corresponds to the intended refractive or vision correction. In the embodiment depicted in FIG. 4A, laser ablation profile 402 is configured to treat myopia. In laser treatments for myopia, the intent is to reduce the curvature, and thus the optical power, of the cornea 410. Accordingly, an ablation profile can confer a reduction in the optical power of the cornea. The curvature of the cornea 410 determines the optical power. Embodiments also include formulating an ablation region AR by an ablation region module, formulating an optical region OR by an optical region module, and/or formulating a transition region TR by a transition module.

Laser energy treatment using the ablation profile 402 shown in FIG. 4A creates a corresponding ablation zone AZ in the cornea 410. Accordingly, profile 402 creates an ablation zone AZ that includes an optically correct central optical zone OZ (corresponding to the central optical region OR), and a transition zone TZ (corresponding to transition region TR). The transition zone TZ is disposed peripherally to the central optical zone. In accordance with a general myopia treatment, the original contour 412 of the cornea has been modified to a lower powered ablated contour 414.

In some embodiments, the central optical region OR has a value within a range from about 3 mm in diameter to about 9 mm in diameter, and in some cases, within a range from about 6 mm in diameter to about 7 mm in diameter.

As shown in FIG. 4A, the diameter d_{AR} of the ablation region AR corresponds to the diameter d_{AZ} of the ablation zone AZ, the diameter d_{OR} of the optical region OR corresponds to the diameter d_{OZ} of the optical zone OZ, the inner perimeter (i.e., inner edge) of the transition region IP_{TR} corresponds to the inner perimeter of the transition zone IP_{TZ}, and the outer perimeter (i.e., outer edge) of the transition region OP_{TR} corresponds to the outer perimeter of the transition zone OP_{TZ}.

According to some embodiments, the transition region TR is used to bring the ablation depth smoothly to zero depth at the ablation profile peripheral edge 404. Transition zone techniques disclosed herein are well suited for implementation in a wide variety of ablation profiles, including arbitrary wavefront shapes.

In refractive surgery, in which human tissue is ablated to correct for vision, a central area (e.g., the optical zone OZ or “the optically correct central optical zone”) of the cornea can be designed to improve the optics within the optical zone OZ for vision. The surrounding area, (e.g., the transition zone TZ) can present undesirable vision problems such as glares, star bursts, and halos, for example due to abrupt curvature changes.

When two surface zones are to be connected and discontinuity between the two zones is a concern, cubic spline functions can be used to transition the ablation surface zone to the unablated cornea surface zone. Cubic spline functions do not adequately address geometric continuity (e.g., a nonzero value to be transitioned to zero) and parametric continuity (e.g., smoothly connecting a slope of the surface (i.e., first derivative)).

The cubic spline functions work well for refractive surgical types such as hyperopia and hyperopic astigmatism. The transition zone TZ can include negative values, however, for conditions such as myopia, myopic astigmatism, and mixed astigmatism when the edge of the optical zone is too steep (i.e., when the slope of the surface is less than −2). Because negative tissue cannot be ablated, however, these negative values are ignored, causing incorrect ablation shapes.

FIG. 4B is a schematic illustration of the example optical zone OZ, example transition zone TZ, and example ablation zone AZ shown in FIG. 4A. FIG. 4B shows a radial line 420 intersecting the center C of the ablation zone AZ and points on the transition zone inner perimeter IP_{TZ} and the transition zone outer perimeter OP_{TZ}. The radial line 420 is a cross section for a corresponding meridian on the surface of the cornea. For cubic spline fitting, the transition zone TZ extends from u=0 to u=1 along any radial meridian, as shown in the example embodiment illustrated in FIG. 4B. Further, because the transition zone function is multiplied to scale the transition zone values, the transition zone function can have values between 0 and 1.

Cubic functions can be used to make a positive edge transition to zero. The general form for a cubic function can be given by:

S(u)=au³+bu²+c·u+d Equation 1

Four equations are used to solve for the four unknowns a, b, c, and d in Equation 1. Assuming that the slope at the outer edge of the optical zone is s. The four conditions that form the four equations are:
When u=0, S(u)=1 for a smooth connection to the inner edge [or geometric continuity at the inner edge);
When u=1, S(u)=0 for a smooth connection to the outer edge (which is 0) [or geometric continuity at the outer edge];
When u=0, S′(u)=s (the slope of S) equals to the connecting slope at the inner edge [or s is parametric (slope) continuity at the inner edge];
When u=1, S′(u)=0 for a smooth slope connection to the outer edge (which is 0) [or 0 is parametric (slope) continuity at the outer edge].

Using the four equations obtained from these conditions yields the spline solution as:

S(u)=(s+2)u³−(2s+3)u²+s·u+1 Equation 2

Here,

$$u = \frac{r - {r}_{in}}{{r}_{out} - {r}_{in}} ,$$

s is the slope au me edge (u=0), r is the radius, rᵢₙ is the inner radius at u=0 or to the treatment radius at the inner periphery, and Rₒᵤₜ is the outer radius at u=1, or the treatment radius at the outer periphery and S satisfies the following conditions:
1. When u=0, S=1 for a smooth connection to the inner edge.
2. When u=1, S=0 for a smooth connection to the outer edge (base line).
3. When u=0, the slope of S is s so the slope has a smooth connection to the inner edge.
4. When u=1, the slope of S is 0 so the slope has a smooth connection to the outer edge.

The slope of S is as follows:

$$\frac{\partial S}{\partial u} = 3 ( s + 2 ) {u}^{2} - 2 ( 2 s + 3 ) u + s \qquad Equation 3$$

When Equation 3 is set to zero, two solutions result, namely, u=1, or

$$u = \frac{s}{3 ( s + 2 )} .$$

Although the above four conditions are met, when the value of u in the second solution is smaller than 1, an undesirable negative run of the spline may occur. That is, the cubic spline function works well when the slope at the inner edge (i.e., inner perimeter) of the transition zone IP_{TZ} is greater than −2. The transition may undesirably run into negative values, however, when s<−2 despite the four conditions given above being satisfied. In reality, negative ablations due not occur. The negative values can be zeroed out to produce the treatment target. Zeroing out these values, however, results in 1) a smaller implemented ablation zone and (2) the first derivative becoming non-zero at the end of the transition zone.

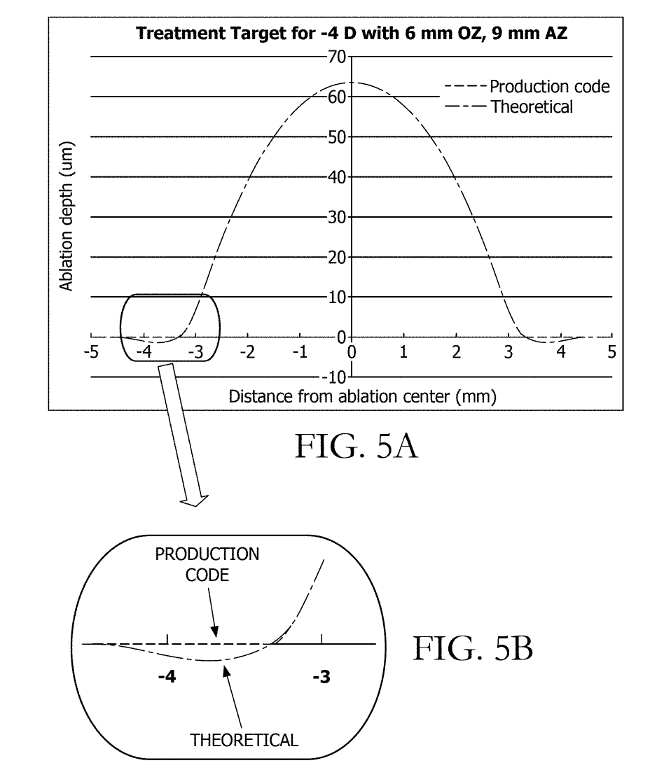

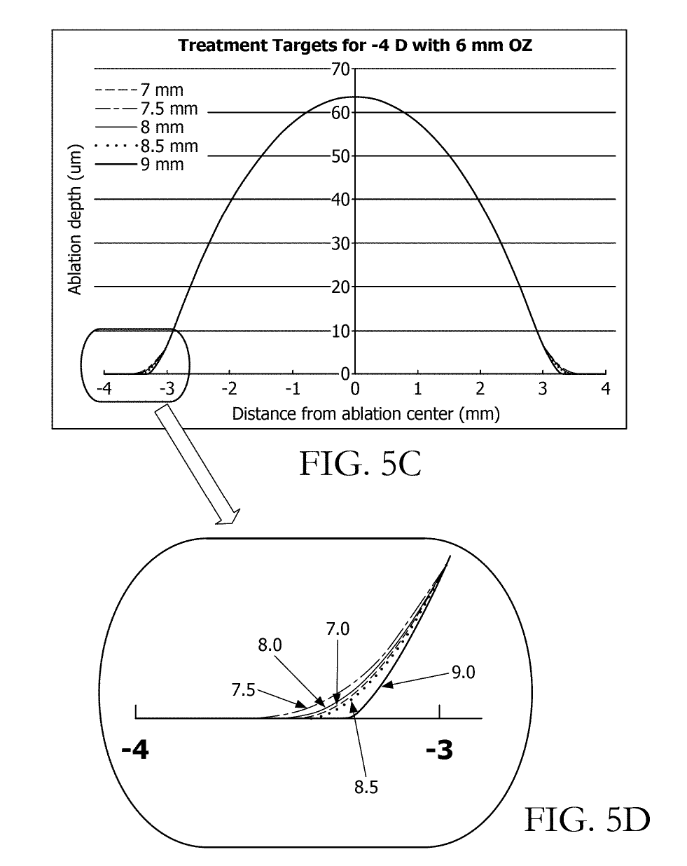

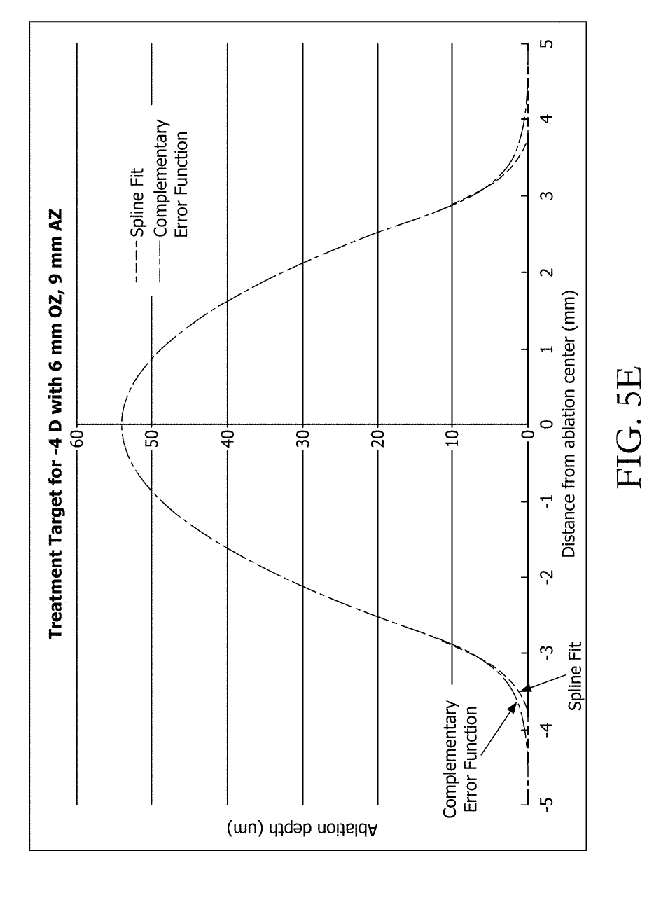

For example, FIG. 5A graphically illustrates a cross section of a −4 D ablation profile using a theoretical calculation using spline fitting and a cross section of a −4 D ablation profile without spline fitting (i.e., production code) for a 6 mm optical zone and a 9 mm ablation zone. FIG. 5B is a blown up view of a portion of FIG. 5 illustrating the comparison between the two cross sections.

The first derivative may have a value of zero at two locations. For example, the first derivative may have a value of zero at r=4.5 mm and r=3.7 mm. Therefore, a U-turn (i.e., negative run or range of negative values) may result. As described above, one solution is to zero out the negative values, so as to produce a production code treatment target. Zeroing out the negative values, however, results in a smaller effective or implemented ablation zone (i.e. less than 7 mm).

For example, with reference to the theoretical ablation profile in FIG. 5A, the theoretical value of the transition zone at u=1 is 0 and the slope at u=1 is also 0. S(u) can be forced to be always positive, as follows:

(s+2)u³−(2s+3)u²+s−u+1≥0

which yields:

$$s \geq - \frac{2 {u}^{3} - 3 {u}^{2} + 1}{{u}^{3} - 2 {u}^{2} + u} \qquad Equation 4$$

It can be shown that the asymptotic value of s is −2:

$${lim}_{u \to ∞} s = {lim}_{u \to ∞} [ - \frac{2 {u}^{3} - 3 {u}^{2} + 1}{{u}^{3} - 2 {u}^{2} + u} ] = - 2 \qquad Equation 5$$

Because the transition zone has negative values, the transition zones are zeroed out, resulting in an effective transition zone equal to 6.5 mm.

In addition, spline fitted transition zone methods may work with myopia profiles only when the ablation zones do not exceed a certain diameter. For example, spline fitting may produce undesirable effects, such as for myopia treatment, when the ablation zone approaches or exceeds a diameter of 7.5 mm to 8 mm.

FIG. 5C is a graphical illustration of cross sections of five exemplary −4 D treatment targets (e.g. wavefront targets), each including an optical zone OZ of 6 mm in diameter and different ablation zones of 7 mm, 7.5 mm, 8 mm, 8.5 mm, and 9 mm in diameter, respectively. FIG. 5D is a blown up view of a portion of FIG. 5 illustrating the comparison between the different cross sections. As shown in FIG. 5C, as the ablation zone AZ increases, the cut-off can decrease, which may be caused by zeroing out the negative values from spline fit algorithm when the end of the optical zone has a slope smaller than −2. For example, a prescribed 9.0 mm ablation zone can effectively render an ablation area that is smaller than an ablation area rendered by a prescribed 7.5 mm ablation zone. Accordingly, undesirable effects may occur for a transition zone for myopia treatment when the ablation zone extends beyond a certain value, such as for example 7 mm.

FIG. 5E is a graphical illustration comparing a cross section of a −4 D ablation profile using spline fitting and a cross section of a −4 D ablation profile using a complementary error function. The complementary error function is utilized to provide transition zone shapes (e.g., curves) for different refractive types, gaining the benefit of analytical expression of its derivatives and non-negativity. While transition zones using spline fitting extend to about 7.5 mm, the complementary error function described below extends the distance between the inner perimeter TZ_{IP} of the transition zone TZ and the outer perimeter TZ_{OP} of the transition zone TZ to 9 mm.

The error function runs asymptotically from 0 to 1 and the complementary error function runs asymptotically from 1 to 0. Accordingly, a transition zone can be provided that approaches zero without running into negative values. In addition, a location of the transition zone can be determined such that the first derivative matches the derivative at the edge of the transition. The complementary error function has the following form:

$$erf c ( u ) = 1 - erf ( u ) = 1 - \frac{2}{\sqrt{π}} {\int }_{0}^{u} {e}^{- {t}^{2}} dt = \frac{2}{\sqrt{π}} {\int }_{u}^{∞} {e}^{- {t}^{2}} dt \qquad Equation 6$$

In addition, the derivative of the complementary error function can be calculated analytically as:

$$\frac{\partial erf c ( u )}{\partial u} = - \frac{2}{\sqrt{π}} {e}^{- {u}^{2}} \qquad Equation 7$$

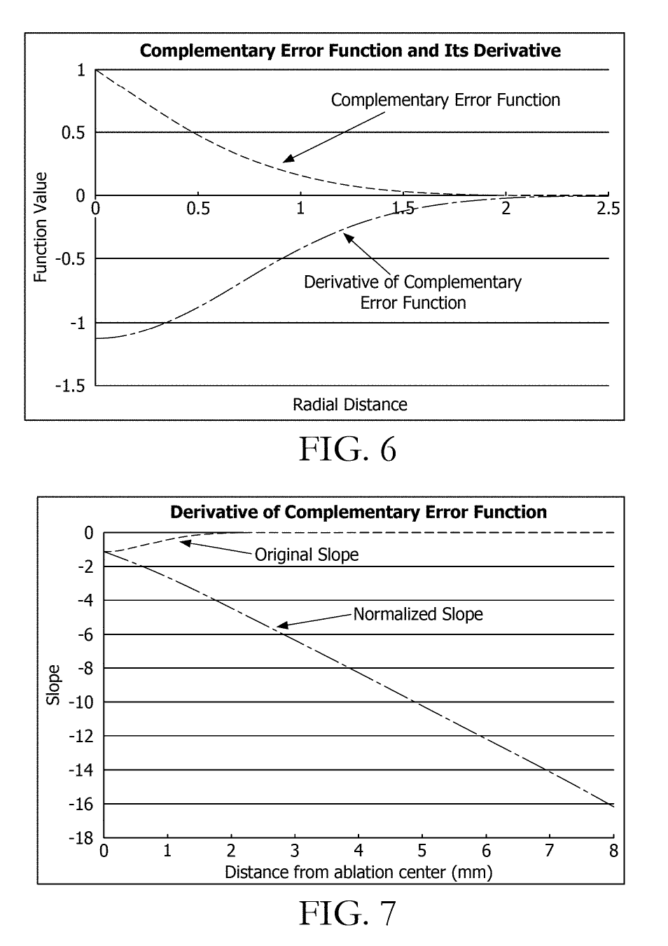

FIG. 6 is a graphical illustration of an exemplary complementary error function and its first derivative for use with embodiments described herein. FIG. 6 shows the change of value of the complementary error function as the radial distance changes. Setting the beginning of the transition zone function to be 1, the complementary error function and its derivative are combined as follows:

$$D ( u ) = \frac{\frac{\partial erf c ( u )}{\partial u}}{erf c ( u )} = - \frac{{e}^{- {u}^{2}}}{{\int }_{u}^{∞} {e}^{- {t}^{2}} dt} \qquad Equation 8$$

The above equation can be approximated as follows:

$$D ( u ) = - \frac{2 u}{\begin{matrix}1 - \frac{1}{2 {u}^{2}} + \frac{1 \cdot 3}{{( 2 {u}^{2} )}^{2}} - \frac{1 \cdot 3 \cdot 5}{{( 2 {u}^{2} )}^{3}} + \\ \frac{1 \cdot 3 \cdot 5 \cdot 7}{{( 2 {u}^{2} )}^{4}} - \frac{1 \cdot 3 \cdot 5 \cdot 7 \cdot 9}{{( 2 {u}^{2} )}^{5}} + …\end{matrix}} \qquad Equation 9$$

By taking a single term in the equation above, an approximation of D(u)=−2u is obtained.

The derivative is normalized by the complementary error function to obtain the normalized derivative of the complementary error function (i.e., the normalized derivative function). A starting value of u₀ is determined by equating D(u) to be the transition slope s. When the starting value u₀ is determined, the remainder of the complementary error function can be used with the same scaling factor for the transition zone function.

Hence, the transition zone function D(u) (i.e., the combination of the complementary error function and the derivative of the complementary error function) can be used to calculate where to start the normalized complementary error function, which is defined as:

$$S ( u ) = \frac{erf c ( {u}_{0} + u )}{erf c ( {u}_{0} )} \qquad Equation 10$$

Then, u₀ can be calculated by using D(u)=s. This property is very useful for the implementation of S(u) as the transition zone function. FIG. 7 is a graphical illustration of normalized and non-normalized derivatives of a complementary error function for use with embodiments described herein. As shown in FIG. 7, the normalized derivative of the complementary error function is substantially a straight line, which facilitates the implementation of S(u) as the transition zone function.

The normalized complementary error function S(u) can have an ending value (u=1) that is too large when multiplied by a large transition zone height. Accordingly, an expanding factor c is introduced, as shown below in Equation 11.

$$c = exp ( \frac{{h}^{2}}{{h}_{0}^{2}} ) \qquad Equation 11$$

where, h is the ablation depth at the transition zone edge and h₀ is a constant determined by experiments. It has been discovered that found that h₀=55 works well for most cases. The final transition zone function can then be determined using the combination of the complementary error function and its derivative and the expanding factor, which is given by the following equation:

$$S ( c \cdot u ) = \frac{erf c ( {u}_{0} + c \cdot u )}{erf c ( {u}_{0} )} \qquad Equation 12$$

Accordingly, the transition zone can be determined by calculating, for each radial meridian across the cornea, a slope at an edge of the optical zone and the transition zone. If the slope is greater than or equal to −2, the cubic spline function is selected to determine the transition zone. If the slope is less than −2, the complementary error function is selected to determine the transition zone.

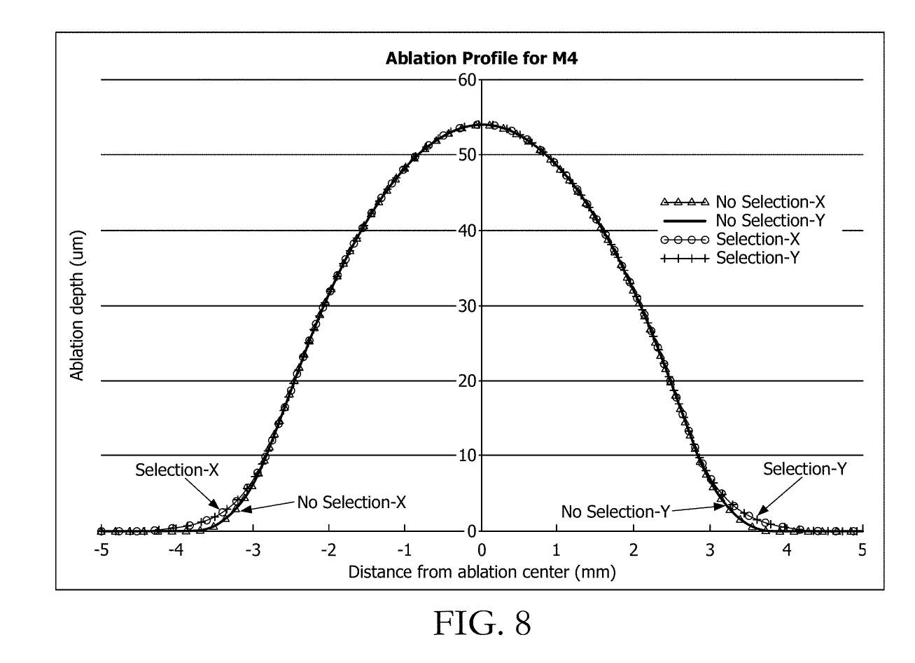

FIGS. 8-10 illustrate comparisons of X and Y cross sections of ablation profiles which select between the spine function and the complementary error function (i.e., “Selection-X” and “Selection-Y”) compared with cross sections which do not select between the spine function and the complementary error function (i.e., “No Selection-X” and “No Selection-Y”).

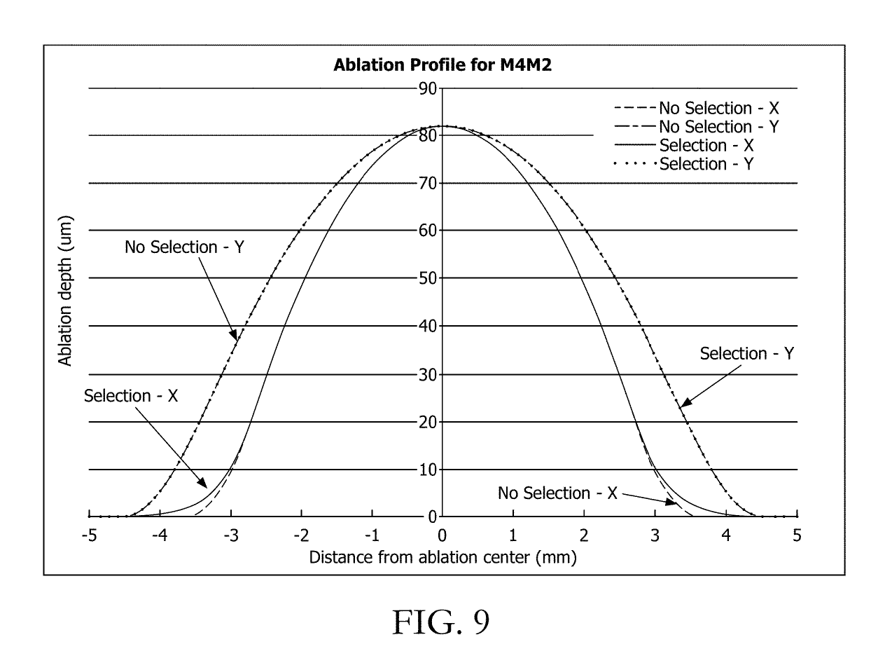

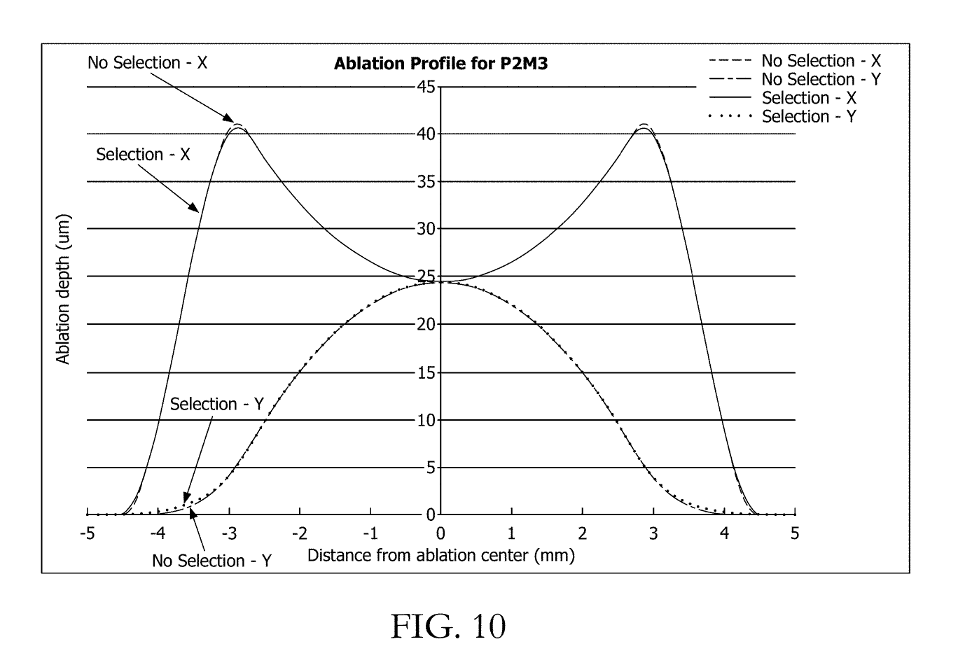

For example, FIG. 8 illustrates X and Y cross-sections of ablation profiles for a −4 D treatment of spherical myopia. FIG. 9 illustrates X and Y cross-sections of ablation profiles for a −4 DS/−2 DC treatment of myopic astigmatism. FIG. 10 illustrates X and Y cross-sections of ablation profiles for a +2 DS/−3 DC treatment of mixed astigmatism. As illustrated in FIGS. 8-10, selecting between the spine function and the complementary error function (i.e., Selection-X and Selection-Y) can avoid the shortcomings (e.g., geometric discontinuity and parametric discontinuity) associated spline function, and the resulting transition zone can reach the target ablation area. For example, by selecting between the spine function and the complementary error function, a transition zone can be provided which reaches out to the 9 mm area.

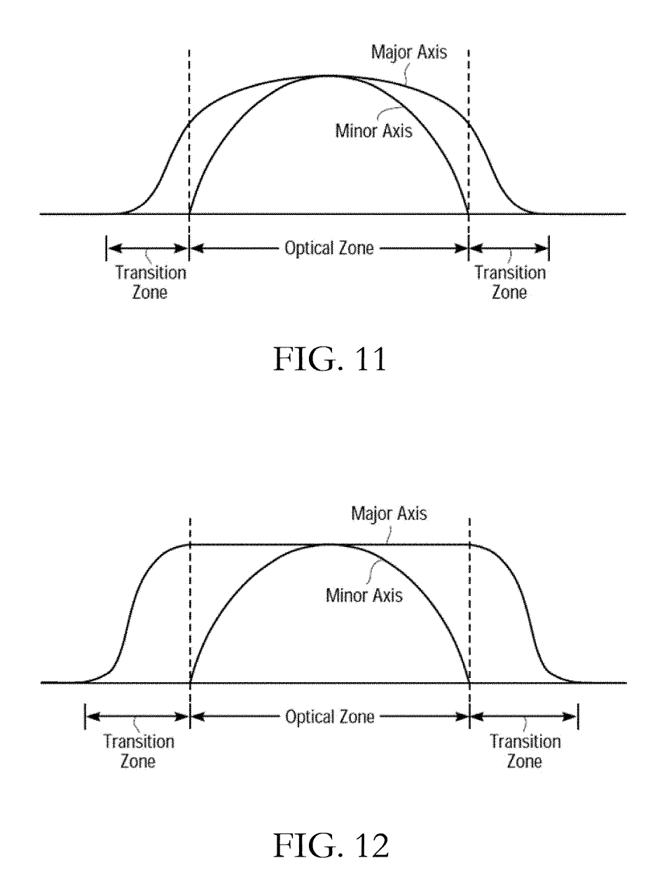

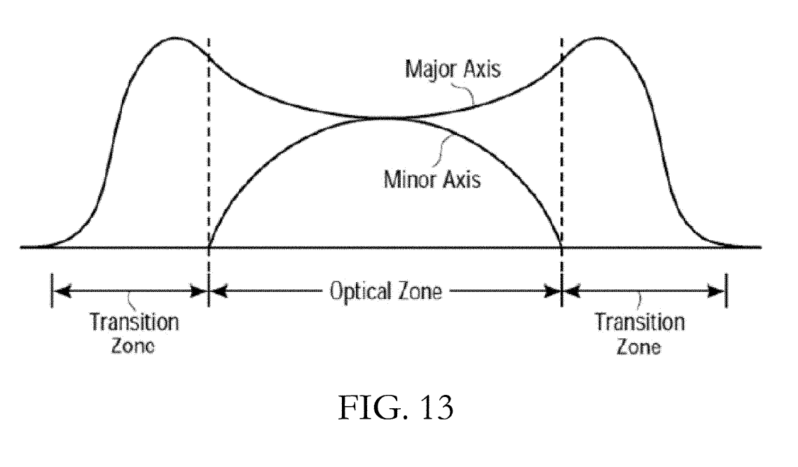

FIGS. 11-13 illustrate different types of exemplary ablation profiles. FIG. 11 illustrates an exemplary ablation profile for a myopic treatment with astigmatism having a transition zone on the major axis, but not on the minor axis. FIG. 12 illustrates an exemplary ablation profile of a cylinder having a transition zone on the major axis but not on the minor axis. The shape in FIG. 12 has a larger transition zone than the transition zone shown in FIG. 11. FIG. 13 illustrates an exemplary ablation profile for a mixed astigmatism treatment shape having a transition zone on the major axis. Embodiments described herein encompass the use of transition zones for myopia, myopic astigmatism, and mixed astigmatism treatments. In some astigmatism embodiments, both the major axis and minor axis can be transitioned to a desired or otherwise appropriate ablation zone.

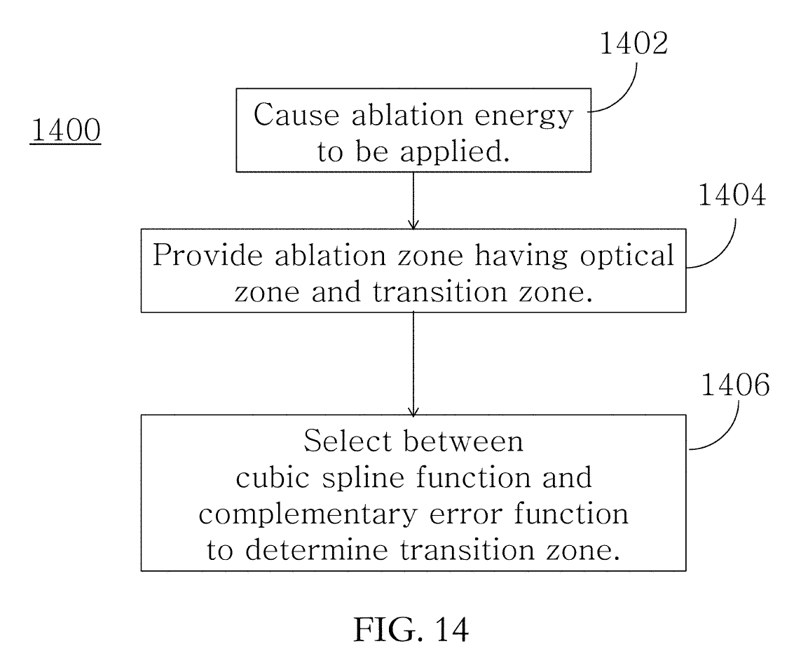

FIG. 14 is a block diagram illustrating an exemplary method 1400 of re-profiling a cornea of an eye. As shown at block 1402, the method 1400 includes causing applying ablation energy to be applied across the cornea of the eye. For example, one or more processors, such as processors 220 may cause an ablation energy source, such as laser 12, to apply ablation energy across the cornea of the eye.

The distribution of the ablation energy is controlled at blocks 1404 and 1406. As shown at block 1404, the distribution of the ablation energy is controlled by causing the ablation energy to provide an ablation zone. For example, laser 12 is controlled to provide an exemplary laser ablation treatment, such as treatment 400 shown in FIG. 4. The ablation energy provides an ablation zone, such as for example, ablation zone AZ shown in FIG. 4, on an anterior surface of the cornea 410, which includes an optical zone OZ disposed in a central portion of the anterior surface, and a transition zone TZ disposed peripherally to the optical zone OZ.

As shown at block 1406, the distribution of the ablation energy is further controlled by determining the transition zone (i.e., parameters of the transition zone). The transition zone is determined by selecting between the cubic spline function and the complementary error function.

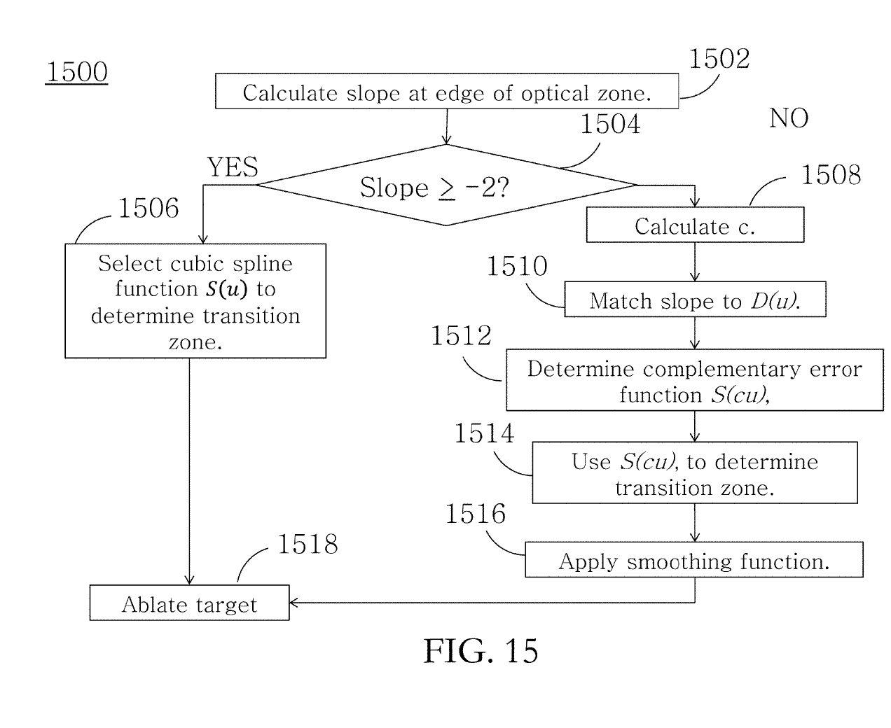

FIG. 15 is a block diagram illustrating an exemplary method 1500 of determining a transition zone at block 1406 of FIG. 14. As shown at block 1502, the method 1500 includes calculating, for one or more radial meridian across the cornea, a slope at the edge of the optical zone and the transition zone (i.e., the inner perimeter of the transition zone IP_{TZ}). In some embodiments, such as ablation treatments which include myopic meridians, values of S(u) for u=0 to u=1 are determined for the one or more radial meridians.

The number of radial meridians used depends on the resolution of the treatment surface. For example, when Cartesian coordinates are used, meridians which correspond to a pixel location are used such that each point is covered. When polar coordinates are used, each of the meridians defined in an angular part is used. Calculations at multiple meridians may be performed in parallel.

As shown at block 1504, the method 1500 includes determining whether the slope is greater than or equal to −2. If the slope is determined at decision block 1504 to be greater than or equal to −2, the method proceeds to block 1506 and the cubic spline function is selected to determine the transition zone.

If the slope is determined at decision block 1504 to be less than −2, the method proceeds to block 1508. S shown at block 1508, the method 1500 includes calculating the expanding constant based on the ablation depth at the transition zone edge. In some embodiments, the expanding constant is based on the ablation depth at the transition zone edge and a constant having a value 3025 (i.e., 55²).

As shown at block 1510, the method 1500 includes matching the slope to the normalized derivative function D(u) to find u₀. As described above with regard to FIG. 7, in one embodiment, an approximation is u₀=−s/(2c).

As shown at block 1512, the method 1500 includes calculating a transition zone function S(cu), using a combination of the complementary error function, its derivative and the expanding factor determined at block 1508. The value of S(cu) is used in place of the original spline function in areas in which the slope is less than −2.

As shown at block 1512, the method 1500 includes using the transition zone function S(cu) to determine the parameters of the transition zone.

As shown at block 1514, the method 1500 includes applying the smoothing function SmoothTZ(t_mesh, r_inner, r_outer) in the transition zone. The smoothing function is, for example, a weighted average of a plurality (e.g., 9) of different points along the radius to smooth out any potential discontinuities.

As shown at block 1518, the target is ablated. For example, corneal tissue of a patient eye may be ablated by laser pulse energy applied by a laser source, such as laser 12.

According to one embodiment, the distribution of the ablation energy (e.g., across the cornea of the eye) includes determining the position of each laser pulse applied by a laser source, such as laser 12, based on a determined shape of the ablation zone and a determined ablation shape from each laser pulse applied by the laser source. The position of each laser pulse applied by the laser source may be determined by using Equation 13 shown below.

$$AblationShape = {\sum }_{n = 1}^{TotalPulses} ( {PulseShape}_{n} ⊗ {Position}_{n} ) \qquad Equation 13$$

Equation 13 is based on the principle that a treatment ablation is the sum of each of the individual laser pulses and can be used to administer the treatment ablation profile. the position of each laser pulse

As shown in Equation 13, parameters of the ablation are defined as AblationShape, PulseShape and Position. AblationShape represents the target ablation shape, PulseShape represents the ablation shape of each laser pulse size to be applied, and Position represents the position of each laser pulse on the target (i.e., corneal tissue).

The AblationShape, which includes the optical region shape and the transition region shape, is predetermined. The optical region shape can be determined by Munnerlyn equations or wavefront techniques. The AblationShape includes, for example, a simple sphere, an ellipse, a cylinder for treating myopia or hyperopia, or a saddle (e.g., for treating mixed astigmatism). The AblationShape can be any arbitrary shape, such as the map from a wavefront type device or any other topography system. The AblationShape can contain, for example, a central optical zone and a transition zone. The AblationShape is a mathematical representation (e.g., theoretical construct) of a predetermined ablation result.

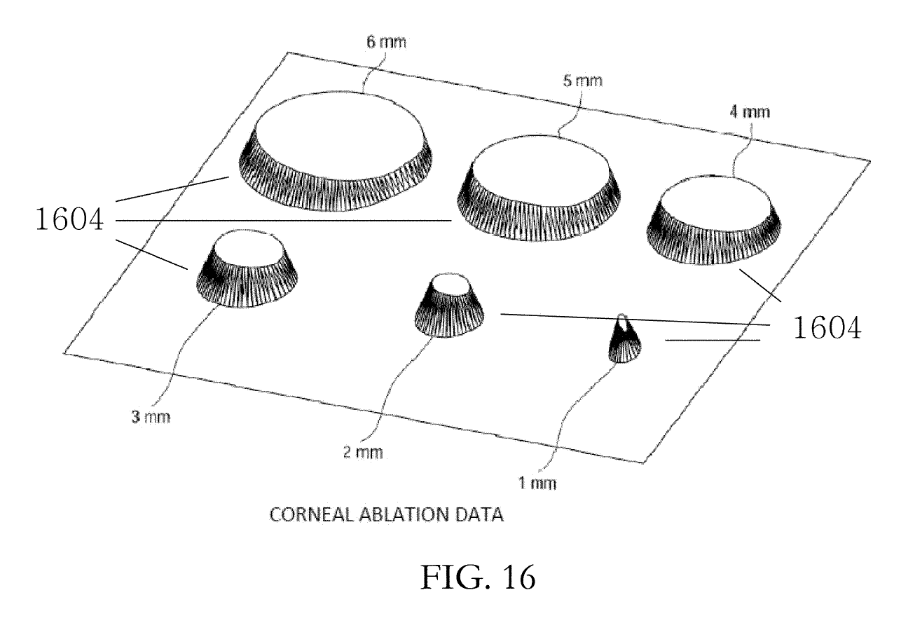

The PulseShape is predetermined and typically varies for different ablated materials, such as plastic, animal cornea, or human cornea and for each laser pulse diameter. FIG. 16 is an illustration of different ablation shapes 1604, having diameters ranging from 1 mm to 6 mm, each of which are caused by a corresponding laser pulse. The size and shape type (i.e. crater) of the shapes 1604 shown in FIG. 16 are merely exemplary. The laser shapes 1604 may be determined prior to implementing an ablation treatment such that a predetermined ablation shape corresponding to one of the ablation shapes 1604 may be used as the AblationShape in Equation 13. There is a unique description for each unique pulse shape or size to be used. The shape ablated onto a specific target material by each laser pulse is measured to generate basis data for a variety of materials, such as tissue or plastic. For a given material at a given diameter, the shape is generally consistent between different laser systems.

Embodiments include using one description (e.g., for a fixed spot laser) of an ablation shape as well as multiple description (e.g., using a variable spot laser of ablation shapes). Embodiments include providing different ablation shapes such as flat shapes, round shapes, and symmetric shapes.

The unknown Position variable, which represents the exact position of each laser pulse, is determined by solving Equation 13 using the predetermined AblationShape and the predetermined PulseShape. For example, processor 220 may determine the PulseShape using Equation 13 and control (e.g., via programmed instructions) a laser source, such as laser 12, to create the target ablation shape using the laser pulses. The programmed instructions may be in the form of a treatment table, which includes of a list of individual pulses, each containing the size and offset, or position, to be used for each corresponding pulse. Based on the instructions in the treatment table, the laser 12 provides the target shape.

Solving for the Position variable facilitates creating an ablation shape to approach the target ablation shape. In this way, each pulse position is individually determined to create the ablation shape to approach the target ablation shape.

Embodiments can also include simulated annealing to facilitate approaching the target ablation shape. For example, one embodiment includes using the simulated annealing least squares (SALSA) algorithm, which solves an equation having over 10,000 unknowns by selecting the number of pulses, the size of each pulse, and the location of each pulse. The SALSA algorithm is an algorithm which makes no statistical assumptions.

Simulated annealing, which is described in PCT Application No. PCT/US01/08337, filed Mar. 14, 2001 and in U.S. Pat. No. 6,673,062, the contents of each being incorporated herein by reference, is a method used to solve intractable problems, and may be used to facilitate the solving of Equation 13. See also W. H. Press et al., “Numerical Recipes in C” 2nd Ed., Cambridge University Press, pp. 444−455 (1992).

All patent filings, scientific journals, books, treatises, and other publications and materials discussed in this application are hereby incorporated by reference for all purposes. A variety of modifications are possible within the scope. A variety of parameters, variables, factors, and the like can be incorporated into the exemplary method steps or system modules. While the specific embodiments have been described in some detail, by way of example and for clarity of understanding, a variety of adaptations, changes, and modifications will be obvious to those of skill in the art. Although embodiments disclosed herein are described with specific reference to a wavefront system using lenslets, other suitable wavefront systems that measure angles of light passing through the eye may be employed. For example, systems using the principles of ray tracing aberrometry, tscherning aberrometry, and dynamic skiascopy may be used with embodiments disclosed herein. The above systems are available from TRACEY Technologies of Bellaire, Tex., Wavelight of Erlangen, Germany, and Nidek, Inc. of Fremont, Calif., respectively. Embodiments may also be practiced with a spatially resolved refractometer as described in U.S. Pat. Nos. 6,099,125; 6,000,800; and 5,258,791, the full disclosures of which are incorporated herein by reference. Treatments that may benefit from the embodiments include intraocular lenses, contact lenses, spectacles and other surgical methods in addition to refractive laser corneal surgery.

All features of the described systems and/or devices are applicable to the described methods mutatis mutantis, and vice versa. Each of the calculations discussed herein may be performed using a computer or other processor having hardware, software, and/or firmware. The methods or flow charts provided herein may be implemented in a computer program, software, or firmware incorporated in a non-transitory computer-readable storage medium for execution by a general purpose computer or a processor. Examples of non-transitory computer-readable storage mediums include a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs).

The methods and apparatuses may be provided in one or more kits for such use. The kits may comprise a system for profiling an optical surface, such as an optical surface of an eye, and instructions for use. Optionally, such kits may further include any of the other system components described in relation to the embodiments described herein and any other materials or items relevant to the embodiments. The instructions for use can set forth any of the methods as described above.

While the above provides a full and complete disclosure of exemplary embodiments, various modifications, alternate constructions and equivalents may be employed as desired. Consequently, although the embodiments have been described in some detail, by way of example and for clarity of understanding, a variety of modifications, changes, and adaptations will be obvious to those of skill in the art. Accordingly, the above description and illustrations should not be construed as limiting the embodiments, which can be defined by the claims.

What is claimed is:

1. A method of re-profiling a cornea of an eye, the method comprising:
causing ablation energy to be applied across the cornea of the eye; and
controlling distribution of the applied ablation energy across the cornea of the eye by:
causing the ablation energy to provide an ablation zone on an anterior surface of the cornea, the ablation zone comprising;
an optical zone disposed in a central portion of the anterior surface, and
a transition zone disposed peripherally to the optical zone; and
determining a shape of the transition zone by selecting between a cubic spline function and a complementary error function.

2. The method according to claim 1, wherein determining the shape of the transition zone further comprises:
calculating, for one or more meridians across the cornea, a slope at an edge of the transition zone adjacent the optical zone;
if the slope is greater than or equal to −2, selecting the cubic spline function to determine the shape of the transition zone; and
if the slope is less than −2, selecting the complementary error function to determine the shape of the transition zone.

3. The method of claim 2, wherein the one or more meridians are myopic meridians.

4. The method of claim 2, wherein determining the transition zone by selecting the complementary error function further comprises:
calculating an expanding constant based on an ablation depth at the edge of the transition zone;
matching the slope at the edge of the transition zone to a normalized derivative of the complementary error function;
calculating a transition zone function using the expanding constant and the normalized derivative function; and
determining the shape of the transition zone using the slope at the edge of the transition zone.

5. The method of claim 4, wherein controlling distribution of the applied ablation energy across the cornea of the eye further comprises modifying the shape of the transition zone by using a smoothing function which comprises determining:
a slope value for each of a plurality of different points along a radius extending from a center of the optical zone; and
a weighted average slope value.

6. The method of claim 1, wherein causing the ablation energy to be applied across the cornea of the eye further comprises causing a laser to apply laser energy across the cornea of the eye.

7. The method of claim 1, wherein controlling distribution of the applied ablation energy across the cornea of the eye further comprises determining a position of each laser pulse applied by the laser based on a determined shape of the ablation zone and a determined ablation shape caused by each laser pulse applied by the laser.

8. A processing device for re-profiling a cornea of an eye, the processing device comprising:
memory configured to store programmed instructions and data; and
a processor in communication with the memory and configured to:
cause ablation energy to be applied across the cornea of the eye; and
control distribution of the applied ablation energy across the cornea of the eye by:
causing the ablation energy to provide an ablation zone on an anterior surface of the cornea, the ablation zone comprising;
an optical zone disposed in a central portion of the anterior surface, and
a transition zone disposed peripherally to the optical zone; and
determining a shape of the transition zone by selecting between a cubic spline function and a complementary error function.

9. The processing device of claim 8, wherein the processor is further configured to:
calculate, for one or more meridians across the cornea, a slope at an edge of the transition zone adjacent the optical zone;
if the slope is greater than or equal to −2, select the cubic spline function to determine the shape of the transition zone; and
if the slope is less than −2, select the complementary error function to determine the shape of the transition zone.

10. The processing device of claim 9, wherein the one or more meridians are myopic meridians.

11. The processing device of claim 9, wherein the processor is further configured to determine the shape of the transition zone by:
calculating an expanding constant based on an ablation depth at the edge of the transition zone;
matching the slope at the edge of the transition zone to a normalized derivative of the complementary error function;
calculating a transition zone function using the expanding constant and the normalized derivative function; and
determining the shape of the transition zone using the slope at the edge of the transition zone.

12. The processing device of claim 11, wherein the processor is further configured to control distribution of the applied ablation energy across the cornea of the eye by modifying the shape of the transition zone using a smoothing function which comprises determining:
a slope value for each of a plurality of different points along a radius extending from a center of the optical zone; and
a weighted average slope value.

13. The processing device of claim 8, wherein the processor is further configured to cause the ablation energy to be applied across the cornea of the eye by controlling a laser source to apply laser energy across the cornea of the eye.

14. The processing device of claim 8, wherein the processor is further configured to control distribution of the applied ablation energy across the cornea of the eye by determining a position of each laser pulse applied by the laser based on a determined shape of the ablation zone and a determined ablation shape caused by each laser pulse applied by the laser.

15. A system for re-profiling a cornea of an eye, the system comprising:
a laser energy source configured to apply laser energy to ablate a cornea of the eye;
a processing device in communication with the laser energy source, the processing device comprising:
memory configured to store programmed instructions and data; and
a processor in communication with the memory and configured to control the laser energy source to cause the laser energy to be applied across the cornea of the eye and control distribution of the applied laser energy across the cornea of the eye by:
causing the laser energy to provide an ablation zone on an anterior surface of the cornea, the ablation zone comprising;
an optical zone disposed in a central portion of the anterior surface, and
a transition zone disposed peripherally to the optical zone; and
determining a shape of the transition zone by selecting between a cubic spline function and a complementary error function.

16. The system of claim 15, wherein the processor is further configured to:
calculate, for one or more meridians across the cornea, a slope at an edge of the transition zone adjacent the optical zone;
if the slope is greater than or equal to −2, select the cubic spline function to determine the shape of the transition zone; and
if the slope is less than −2, select the complementary error function to determine the shape of the transition zone.

17. The system of claim 16, wherein the one or more meridians are myopic meridians.

18. The system of claim 16, wherein the processor is further configured to determine the shape of the transition zone by:
calculating an expanding constant based on an ablation depth at the edge of the transition zone;
matching the slope at the edge of the transition zone to a normalized derivative of the complementary error function;
calculating a transition zone function using the expanding constant and the normalized derivative function; and
determining the shape of the transition zone using the slope at the edge of the transition zone.

19. The system of claim 18, wherein the processor is further configured to control distribution of the applied laser energy across the cornea of the eye by modifying the shape of the transition zone using a smoothing function which comprises determining:
a slope value for each of a plurality of different points along a radius extending from a center of the optical zone; and
a weighted average slope value.

20. The system of claim 15, wherein the processor is further configured to control distribution of the applied laser energy across the cornea of the eye by determining a position of each laser pulse applied by the laser based on a determined shape of the ablation zone and a determined ablation shape caused by each laser pulse applied by the laser.
